(19) United States
(12) Patent Application Publication
(10) Pub. No.: US 2008/0131405 A1
(43) Pub. Date: Jun. 5, 2008

(54) COMPOSITION FOR TREATING A DISEASE CAUSED BY NEURONAL INSULT COMPRISING A HUMAN UMBILICAL CORD BLOOD-DERIVED MESENCHYMAL STEM CELL AS AN ACTIVE INGREDIENT

(76) Inventor: Sin Soo Jeun, Kyungki-do (KR)

Correspondence Address: THELEN REID BROWN RAYSMAN & STEINER LLP, P.O. BOX 640640, SAN JOSE, CA, 95164-0640, US

(21) Appl. No.: 11/755,747

(22) Filed: May 31, 2007

(30) Foreign Application Priority Data
Nov. 30, 2006 (KR) 10-2006-0120227

(51) Int. Cl. A61K 35/00; A61P 25/00

(52) U.S. Cl. 424/93.7

(57) ABSTRACT

Provided is a composition for treating nerve damage-related diseases. The composition includes a human umbilical cord blood-derived mesenchymal stem cell as an active ingredient. The mesenchymal stem cell isolated and incubated from the human umbilical cord blood migrates to an injured area to be differentiated into a nerve cell or a neuroglial cell at the time of in vivo transplantation. Thus, the mesenchymal stem cell and a composition including the same can be effectively used in cell replacement therapy and gene therapy for treating diseases caused by nerve damage including a stroke, Parkinson's disease, Alzheimer's disease, Pick's disease, Huntington's disease, amyotrophic lateral sclerosis, traumatic central nervous system disease and a spinal cord injury.

### CROSS-REFERENCE TO RELATED APPLICATION

This application claims the benefit of Korean Patent Application No. 2006-0120227, filed Nov. 30, 2006, the contents of which are hereby incorporated herein by reference in their entirety.

### BACKGROUND OF THE INVENTION

1. Field of the Invention

The present invention relates to a composition for treating nerve damage-related diseases comprising a human umbilical cord blood-derived mesenchymal stem cell as an active ingredient.

2. Description of the Related Art

Traumatic spinal cord injury, one type of injury resulting in difficult-to-cure damage to the nervous system, produces an excitotoxicity neurotransmitter¹, a free radical², an inflammation promoting medium³, and so forth. These give rise to apoptosis of nerve cells and neuroglial scars are formed, which creates an environment that inhibits nerve regeneration. Treatment for such spinal cord injury focuses on development of material which activates genes associated with nerve regeneration or suppresses generation and functions of neuroglial scars inhibiting nerve regeneration. Drug treatment using nerve growth factors or the like and cell treatment using stem cells are actively done.

The mesenchymal stem cell used in stem cell treatment has a multi-differentiation¹⁻³ ability to various tissues, and enables easy isolation from marrow human umbilical cord blood³⁻⁸, and adipose tissue.

Research using marrow as a main origin of the mesenchymal stem cell⁴⁻⁸ has progressed considerably. However, recent studies have brought to light the effects of using human umbilical cord blood-derived mesenchymal stem cells to treat obstinate nerve system diseases in animals, and thus hopes are high for using human umbilical cord blood-derived mesenchymal stem cells as an effective cell treatment⁹⁻¹⁰. In the case of spinal cord injury, it was reported that the transplanted stem cell migrates to the injured area and represents glia or neurogenous phenotype⁴⁻¹². According to such research, when the mesenchymal stem cells were transplanted to the injured spinal cord area, it is reported that these cells played the role of a bridge connecting the injured areas, and enhanced nerve impulse conduction to induce functional recovery from spinal cord paralysis.

A variety of research for enhancing a transplantation effect of mesenchymal stem cells has recently been conducted, and one of line of research is related to a transplantation method. The stem cell transplantation method includes a direct transplantation method at the epicenter, rostral, or caudal of an injured area, and an indirect transplantation method through a fourth ventricle¹³⁻¹⁶ or lumbar puncture²¹⁻²⁴.

However, it is reported that, cell engraftment and survival ability may be degraded by a micro-environment of the injured area when the cells are directly transplanted to the epicenter of the injury, and secondary injury may occur when the cells are transplanted to the rostral.

In addition, it is reported that it is difficult to implement cell engraftment or cell survival and migration to the injured area due to an already broken nerve path when the cells are transplanted to the caudal of the injured area.

### SUMMARY OF THE INVENTION

It is therefore an object of the invention to provide a highly safe means for treating nerve damage-related diseases and a composition for the same. More particularly, the invention aims to provide a composition administered in vivo for treating nerve damage-related diseases. The composition is to include a mesenchymal cell, particularly a human umbilical cord blood cell, a marrow cell, a peripheral blood cell, or a mesenchymal stem cell derived from the cell as an active ingredient, and be suitable for rostral administration to the nerve damage area.

The present inventors, who have conducted research toward the above objectives, checked for therapeutic effects on nerve damage-related diseases by inducing spinal cord injury in mice, sampling human umbilical cord blood, separating mesenchymal stem cells only from the sampled blood, and in vivo administering the mesenchymal stem cells to the injured mice as donor cells.

It was surprisingly found that in vivo administration of the human umbilical cord blood-derived mesenchymal stem cell has a therapeutic effect on nerve damage-related diseases (stroke, spinal cord injury disease).

Particularly, it was confirmed that, when the cell is transplanted to the rostral area of the injured area, rather than the epicenter and caudal areas, behavioral motor ability recovery was better and migration to the lesion area and engraftment of the transplanted cells were higher.

As described above, the present inventors have completed the present invention by confirming the therapeutic effect on nerve damage-related diseases by means of in vivo administration of the mesenchymal cells, particularly, the human umbilical cord blood stem cell. The present inventors have analyzed in detail and proved the therapeutic effect on nerve damage-related diseases by means of in vivo administration of the mesenchymal stem cells in medical or biological experiments.

That is, it is inferred that the mesenchymal cell, particularly, the human umbilical cord blood-derived mesenchymal stem cell is a viable in vivo-administered composition for nerve damage-related diseases. Thus, it is expected that the human umbilical cord blood-derived mesenchymal stem cell will become a nerve damage protective agent or a nerve damage regenerating agent for in vivo administration.

The present invention relates to a composition administered in vivo for treating nerve damage-related diseases, the composition including a mesenchymal cell, particularly, a human umbilical cord blood cell, a marrow cell, a peripheral blood cell, or a mesenchymal stem cell derived from the cell as an active ingredient, and suited for rostral administration to the nerve damage area.

In addition, the present invention relates to a composition administered in vivo and having a nerve damage protection effect or a nerve damage regeneration effect, the composition including the mesenchymal cell as an active ingredient, a use of the component, and a treatment method thereof.

### BRIEF DESCRIPTION OF THE DRAWINGS

The foregoing and other objects, features and advantages of the invention will become apparent from the following more particular description of exemplary embodiments of the invention, as illustrated in the accompanying drawings. The drawings are not necessarily to scale, emphasis instead being placed upon illustrating the principles of the invention.

FIG. 1 is a graph illustrating BBB grades before and after human umbilical cord blood-derived mesenchymal stem cells (hUCB-MSCs) are transplanted to a rat having a spinal cord injury.

FIG. 2 illustrates a cavity shape within a rat having a spinal cord injury four weeks after hUCB-MSCs transplantation. Formed cavities were much smaller and narrower in groups B, C, and D transplanted with hUCB-MSCs than in the control group, and were particularly smaller and narrower in group D where the hUCB-MSCs were transplanted at the rostral.

FIG. 3 is a graph illustrating cavity volume measurements at the fifth week of injury, wherein the cavity volumes are reduced in the groups of the present invention, cavities are small and narrow in groups B, C, D, and particularly, group D transplanted at the rostral had smaller and narrower cavities than groups B and C (*P<0.05) as a result of measurement at the fourth week after transplantation.

FIG. 4 illustrates distributions of the hUCB-MSCs labeled with PKH26 in the spinal cord injury at the fourth week after transplantation. It can be seen that the PKH26-labeled cells survive to be distributed mainly around the injured area of the spinal cord. Groups transplanted to the epicenter are A, D, G, groups transplanted to the caudal are B, E, H, and groups transplanted to the rostral are C, F, I. PKH26 (red fluorescence), DAPI (blue fluorescence)

FIG. 5 illustrates a method of isolating a human umbilical cord blood-derived mesenchymal stem cell.

FIG. 6 is a graph illustrating BBB grades using SCI before and after transplantation of the human umbilical cord blood-derived mesenchymal stem cell.

FIG. 7 is an immunohistofluorescence micrograph illustrating an immunity stain with respect to the GFAP (green fluorescence).

FIG. 8 is a double-labeled microphotograph with respect to the PKH26/nerve combined marker.

FIG. 9 illustrates quantitative analysis results of cell density within a white portion.

FIG. 10 is a graph illustrating a distribution pattern of individual glial phenotype within a pool of a white BrdUrd at the 14^{th} day after transplantation.

FIG. 11 shows that transplantation of the human umbilical cord blood-derived mesenchymal stem cell increases synthesis of nerve factor expression within the traumatic spinal cord nerve damage area.

FIG. 12 is a cross-sectional view illustrating a device used for an in vitro migration assay.

FIG. 13 illustrates an MCAO adhesive-removal test (rotarod test) result.

FIG. 14 illustrates the number of UCB-MSCs migrated to the MCAO brain tissue extracts at the seventh day after ischemia.

FIG. 15 shows that time serial detection UCB-MSCs migrate to the infarction lesion.

### DETAILED DESCRIPTION OF THE INVENTION

The present invention will now be described more fully hereinafter with reference to the accompanying drawings, in which exemplary embodiments of the invention are shown.

The present invention provides a composition administered in vivo for treating nerve damage-related diseases, the composition including a mesenchymal cell (e.g., a human umbilical cord blood cell, a marrow cell, a peripheral blood cell, or a derived mesenchymal stem cell derived from the cell) as an active ingredient, and suited for rostral administration to the nerve damage area.

In the present invention, in vivo administration typically means administration to the injured area. For example, epicenter administration, rostral administration, caudal administration or the like may be employed, however, the most preferred mode of administration is rostral.

In the present invention, a human umbilical cord blood-derived mesenchymal stem cell is usually used. The present inventors have already confirmed that, when a mesenchymal stem cell formed by fractionation of a mononuclear cell separated from human umbilical cord blood is incubated in a basal incubation solution, the cell is induced and differentiated to a nerve cell or neuroglial cell. At this time, the basal incubation solution is not particularly limited, however, Dulbecco's modified essential medium (DMEM) is preferably an Neural progenitor cell basal medium (NPBM)(SClonetics). Components other than the basal incubation solution are not particularly limited, however, F-12, FCS, neural survival factors (Clonetics) may be preferably employed. The concentration of such an incubation solution is, for example, 50% for F-12 and 1% for FCS. In addition, the CO₂ concentration of the incubation solution is preferably 5% but is not limited thereto.

Cell fractionation of the present invention is performed such that 900 g of a human umbilical cord blood cell sampled from a vertebrate animal is subjected to density gradient centrifugation in a solution for a time sufficient for isolation corresponding to the specific gravity. Then, cell fractionates are recovered with a constant specific gravity included in a range from 1.07 g/ml to 1.1 g/ml, and can thus be compounded. In this case, the time sufficient for isolation corresponding to the specific gravity means a sufficient time for the cell to occupy a position corresponding to the specific gravity in the solution for the density gradient centrifugation, and is typically 10 to 30 minutes. The specific gravity of the recovered cell fractionates is preferably in the range from 1.07 g/ml to 1.08 g/ml (e.g., 1.077 g/ml). A Ficol solution or a Percol solution may be used as the solution for density gradient centrifugation, however the solution is not limited thereto.

In addition, an active ingredient of a composition for treating nerve damage-related diseases for in vivo administration may include, for example, a human umbilical cord blood cell, a marrow cell, and the cell fractionate. The mesenchymal cell of the present invention, e.g., the human umbilical cord blood cell, and the marrow cell, may be used for administration in itself. However, in order to enhance treatment efficiency by means of administration, a composition of several medical agents or a cell with genes functioning to enhance therapeutic effect may be taken into consideration.

Fabrication of a transgenic cell or a composition of the present invention may include:

(1) addition of a material promoting differentiation into a nerve cell, enhancing proliferation rate of a cell included in a cell fractionate, or introduction of genes having such an effect.

(2) addition of a material enhancing a survival rate within injured nerve tissue of a cell included in a cell fractionate, or introduction of genes having such an effect.

(3) addition of a material inhibiting an adverse effect on a cell included in a cell fractionate due to injured nerve tissue, or introduction of genes having such an effect.

(4) addition of a material lengthening the life of a donor cell, or introduction of genes having such an effect.

(5) addition of a material adjusting a cell cycle, or introduction of genes having such an effect.

(6) addition of a material suppressing immunity reaction, or introduction of genes having such an effect.

(7) addition of a material making energy metabolism active, or introduction of genes having such an effect.

(8) addition of a material having nerve protection action, or introduction of genes having such an effect.

(9) addition of a material having a apoptosis suppressing effect, or introduction of genes having such an effect.

The mesenchymal cell introduced in a state of expressing preferred genes can be properly fabricated by using a technique that is well known in the art.

A composition for in vivo administration for treating nerve damage-related diseases including a mesenchymal cell of the present invention as an active ingredient can be fabricated by a method well known in the art. For example, it can be used as a type of water-based pharmacologically acceptable aseptic solution, or a type of injected suspension solution if necessary. For example, it may be properly combined with a pharmacologically acceptable carrier or medium, particularly, sterilized water or a physiological salt solution, plant oil, an emulsifying agent, a suspending agent, a surface active agent, a stabilizer, an excipient, a vehicle, an antiseptic, a bonding agent, thereby fabricating an agent by mixing in a unit capacity form generally required for manufacturing medicine. Referring to the manufacture, the amount of active ingredient is intended to have a proper capacity within an instructed range. In addition, an aseptic composition for injection may be prescribed in accordance with typical manufacturing using a vehicle such as distilled water for injection.

In this case, examples of a water solution for injection may include a physiological salt solution, an isotonic solution containing glucose or other adjuncts, for example, D-sorbitol, D-mannose, sodium chloride, and may be used in combination with a proper dissolution adjuvant (compound), e.g., alcohol, particularly, ethanol, polyalcohol, e.g., propylene glycol, polyethylene glycol, non-ionic surface active agent, e.g., polysorbate 80(TM), HCO-50.

Sesame oil, bean oil or the like may be used as an oil solution, and that may be used in combination with Benzyl benzoate or benzyl alcohol as a solution adjuvant. In addition, it may be combined with a buffer such as phosphate buffer solution, sodium acetate buffer solution, an analgesia agent such as chloroprocaine, a stabilizer such as benzyl alcohol, phenol, and an antioxidant. The compounded injection solution is usually charged in a suitable ample.

In vivo administration for patients is preferably parenteral administration. Particularly, administration to an injured area is basically one time, but may be several times. In addition, the administration time may be long or short. More particularly, an injector type, a transdermal administration type or the like may be employed.

Nerve damage-related diseases of the present invention may be, for example, stroke, Parkinson's disease, Alzheimer's disease, Pick' disease, Huntington's disease, amyotrophic lateral sclerosis, traumatic central nervous system disease and spinal cord injury, however, particularly stroke or spinal cord injury.

In addition, in the treatment of the present invention, administration of the composition of the present invention to patients may be very suitably carried out in accordance with the above-described method. In addition, the method may be suitably modified by a doctor to administer the composition to patients.

In addition, the above-described treatment of the present invention is necessarily not limited to humans. It may be applied to mammals as well (e.g., mice, rats, rabbits, pigs, dogs, monkeys, etc.).

Hereinafter, the present invention will be described more fully with reference to exemplary embodiments. These embodiments are only illustrative, and the present invention is not limited thereto.

### EMBODIMENTS

### First Embodiment

### Experiment using a Spinal Cord Injury Model of White Rat

1. Material and Method

1.1. Separation of Human Umbilical Cord Blood-Derived Mesenchymal Stem Cell

Human umbilical cord blood obtained with parental consent were separated by Ficol gradient (density 1.077 g/cm³, Sigma Co.) to clean mononuclear cells, which were then suspended by α-MEM (Gibco BRL) contained with 10% FBS (HyClone) and divided at a concentration of 5×10⁶ cells/cm². An incubating solution was exchanged two times a week, and the cells were incubated with 5% CO₂ at 37° C.

When incubation of mononuclear cells derived from the human umbilical cord blood was established and fibro blast like adhesive cells were observed, 0.25% Trypsin (HyClone) was processed to separate cells and then was suspended again by an incubating solution when a monolayer of colonies was 80% after three weeks.

After the cells were secondarily incubated with a dimension of 5×10⁴ cells/cm², they were in vitro expanded simultaneously while differentiation potential was studied.

1.2. Spinal Cord Injury Model

A white male rat of 270+5 g was injected with Ketamin (80 mg/kg) and Xylazine (10 mg/kg) at its abdominal cavity and then paralyzed. The paralyzed white rat was incised on its back and T9 was subjected to laminectomy at T8 to expose the spinal cord, an impact bar of an NYU impactor was put on T9 to set a reference line, and then the spinal cord was dropped at a height of 25 mm to induce an unstable spinal cord injury.

In order to prevent inflammation of the operation area, Gentamicin (30 mg/kg/day) as an antibiotic was intramuscular injected for seven days and urine was removed twice every day to prevent bladder rupture.

1.3. Transplantation of Human Umbilical Cord Blood-Derived Mesenchymal Stem Cell

After spinal cord injury models having a height of 25 mm were made by an NYU impactor, models having about 4 points of BBB score were selectively determined in one week and then divided into four groups to be transplanted with a human umbilical cord blood-derived mesenchymal stem cell per area.

Group 1 was injected with PBS as a control group of this experiment, group 2 was transplanted with a human umbilical cord blood-derived mesenchymal stem cell labeled with PKH26 to a depth of 3 mm at the rostral 5 mm away from the epicenter of the injury. Group 3 was directly transplanted at the epicenter of the injury, and group 4 was transplanted at the caudal 5 mm away from the epicenter of the injury.

1.4. Behavioral Test

Motor ability recovery was evaluated by Basso, Beattie, and Bresnahan (BBB) scores for five weeks after spinal cord injury.

The BBB scores are composed of 21 points and mean scores on movement of rear legs, treading the ground, leg angles, body stability, and so forth. Scoring was carried out by two observers not taking part in the experiment, fully aware of the method of scoring basic points only, and observing movements of the rats regardless of the test group and the control group.

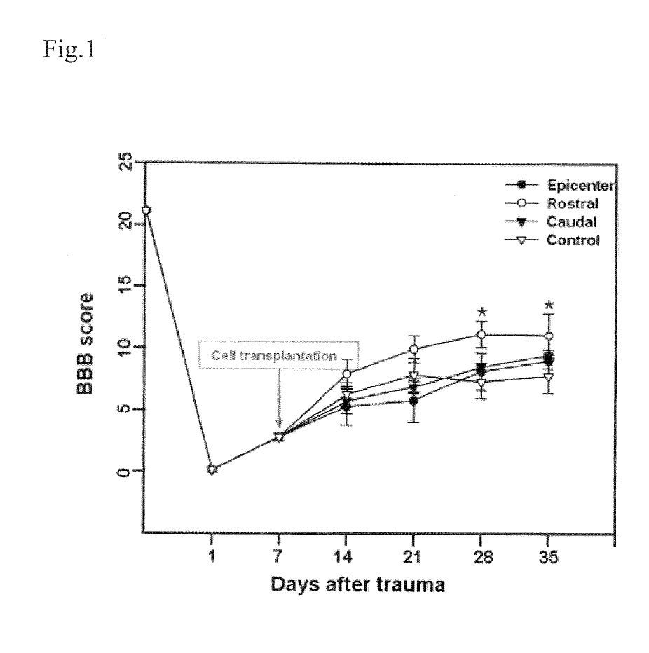

To detail this, on the first day and first, second, third, fourth, and fifth weeks after spinal cord injury, behavioral changes in lower limbs of 30 white rats were observed using the above-described BBB scoring method. The results are shown in FIG. 1.

As seen in FIG. 1, the control group subjected to PBS gradually improved so that the BBB score at the fifth week was 6.5±0.2. In contrast, groups transplanted with the human umbilical cord blood-derived mesenchymal stem cells had a significantly improved motor ability recovery from the third week after injury compared to the control group subjected to PBS (P<0.05). And among these groups, the group transplanted at the rostral had a better behavioral movement than the groups transplanted at other areas.

In particular, scores of the groups transplanted with the cell at the fifth week were 10.5±1.3 in group 2, 9.1±0.6 in group 3, and 9.3±0.4 in group 4.

Among these rats, rats having low BBB scores (not greater than 4) and having similar injury degrees on both lower limbs were selectively used for transplanting PBS or the human umbilical cord blood-derived mesenchymal stem cells.

1.4. Perfusion Fixation and Preparation of Tissue Sample

At the fourth week after transplantation of the human umbilical cord blood-derived mesenchymal stem cell, rats of each group were strongly paralyzed by chloral hydrate (350 mg/kg) and their blood was cleaned with PBS, perfusion fixation was carried out thereon using a 4% paraformaldehyde (PFA) solution, and the operation area was incised to obtain spinal cords, which were stored for a day at 4° C. in 4% PFA.

The next day, they were sequentially put into each of 10%, 20%, 30% sugar solutions dissolved in a 0.1M phosphoric buffer (PB) solution and left until they completely sank to the bottom in each stage, which was then embedded by OCT using liquid nitrogen (LN₂). The embedded tissue was stored at −80° C.

1.5. Immunohistochemistry.Fluorescent Staining

Tissue stored at −80° C. was continuously cut centered on the injured area to a thickness of 10 μm by a freezing microtome, and then was attached to a slide.

The slide was then cleaned by 0.01M PBS for 10 minutes, and first antibodies were treated at 4° C. after it was blocked in a 10% goat serum containing 0.3% tripton X-100. In this case, antineuronal class III β-tublin (TUJ1, 1:100, Chemicon), anti-neuronal nuclei (NeuN, 1:100, Chemicon), anti-2,3′-cyclic nucleotide 3′-phosphodiesterase (CNPase, 1:100, Chemicon), anti-myelin basic protein (MBP, 1:100, Chemicon), anti-glial fibrillary acidic protein (GFAP, 1:200, Chemicon) were used as the first antibodies.

It was then cleaned with 0.01M PBS, and a second antibody, Alexa 488-conjugated goat anti mouse IgG (1:200, Vector Laboratories), was treated for one hour.

It was cleaned with PBS and then DAPI (1:1000, Sigma) was treated. It was rinsed by 0.1M PB, sealed by a cover glass, and checked by a fluorescent microscope.

1.6. Measurement of Cavity Volume

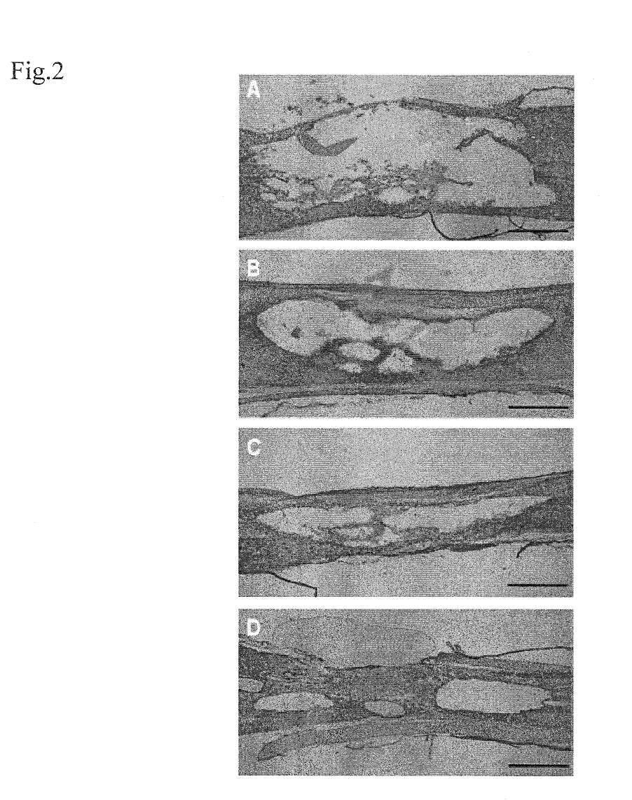

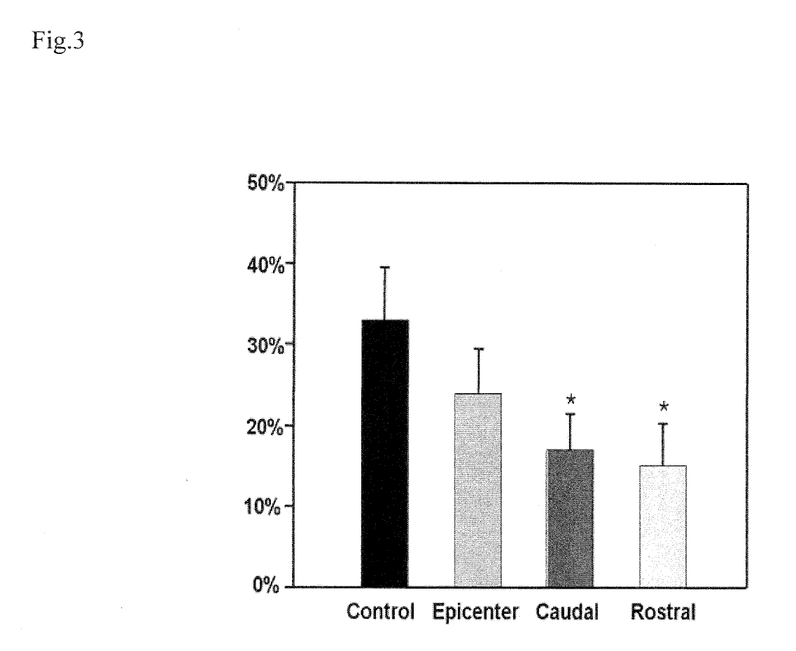

Orthogonal slide sections were observed by a light microscope using a CCD camera, hematoxylin & eosin (H&E), and an immunohistochemisry staining in order to measure cavity volumes. Results obtained by the immunohistochemisry staining are shown in FIG. 2. Cavity volumes of injured areas between groups were measured by the H&E method and compared in FIG. 3.

As can be seen in FIG. 2, it was confirmed that many astrocyte cells are present which react with GFAP in cavities of groups transplanted with the human umbilical blood-derived mesenchymal stem cells. In particular, it could be found in FIG. 2 that many astrocyte cells are present, which react with GFAP in group 2 transplanted at the rostral of the injured area.

Meanwhile, referring to FIG. 3, measurements of the cavity volume were calculated by means of Cavalier's correction by combining cavities of the spinal cord slices. All data were represented as a percentage or a standardized volume (±(Standard error of the mean (SEM)) of the spinal cord volume.

As a result, as can be seen from FIG. 3, it was confirmed that the cavity size of the control group treated with PBS at five weeks after spinal cord injury was 33%, and the cavity volume was reduced when the cavity volume of the groups transplanted with the human umbilical cord blood-derived mesenchymal stem cells was compared with the comparative group treated with PBS. Actually, the cavity volume was 15% in group 2, 24% in group 3, and 17% in group 4.

It can be seen that group 2 transplanted at the rostral of the injured area among the groups had a smaller cavity than groups transplanted at other areas.

1.7 Statistical Processing

A behavioral test between the transplantation group and the control group, and a test of measuring cavity volumes, were performed by means of t-test to verify significance, which was accepted when the P value is less than 0.05.

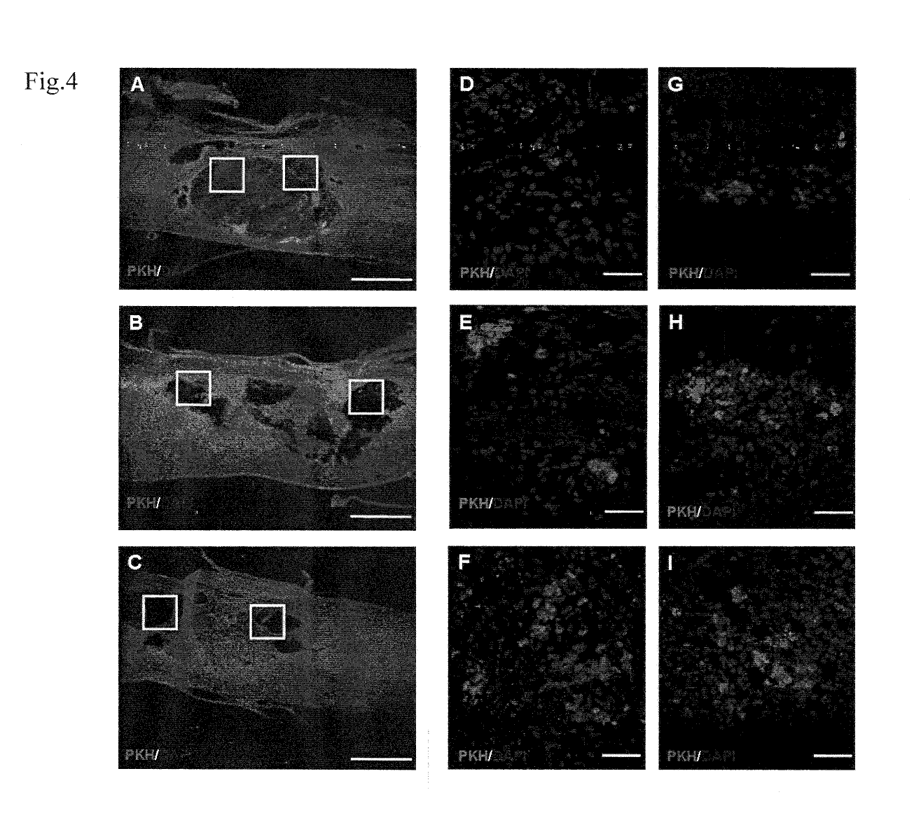

In order to check whether the human umbilical cord blood-derived mesenchymal stem cells are engrafted, it was stained with DAPI and measured by a fluorescent microscope and the result is shown in FIG. 4. As a result, human umbilical cord blood-derived mesenchymal stem cell labeled with PKH26 in each group was found near the injury are at the fourth week after transplantation

It can be found from FIG. 4C that many human umbilical cord blood-derived mesenchymal stem cells labeled with PKH26 are at rostrals of groups among groups transplanted with the human umbilical cord blood-derived mesenchymal stem cells. In addition, it was confirmed that most mesenchymal stem cells transplanted at the rostral area and surviving at a region around the lesion were differentiated into mature astrocyte cells and some cells were differentiated into nerve cells.

### Second Embodiment

### Effect of Human Umbilical Cord Blood-Derived Mesenchymal Stem Cell on Endogenous Neurogenesis of Spinal Cord Injury of Contused Mature White Rat

In the present embodiment, it was checked 1) whether the transplanted hUCB-derived MSCs can improve a function outcome of the mature rat having the contusive spinal cord injury (SCI), 2) whether the hUCB-derived MSCs can be differentiated and expressed into a nerve or neuroglial cell and 3) whether the hUCB-derived MSCs derive the expression of the neurotrophic facto regenerated after SCI or proliferation of intrinsic endogenous neural stem/progenitor cells.

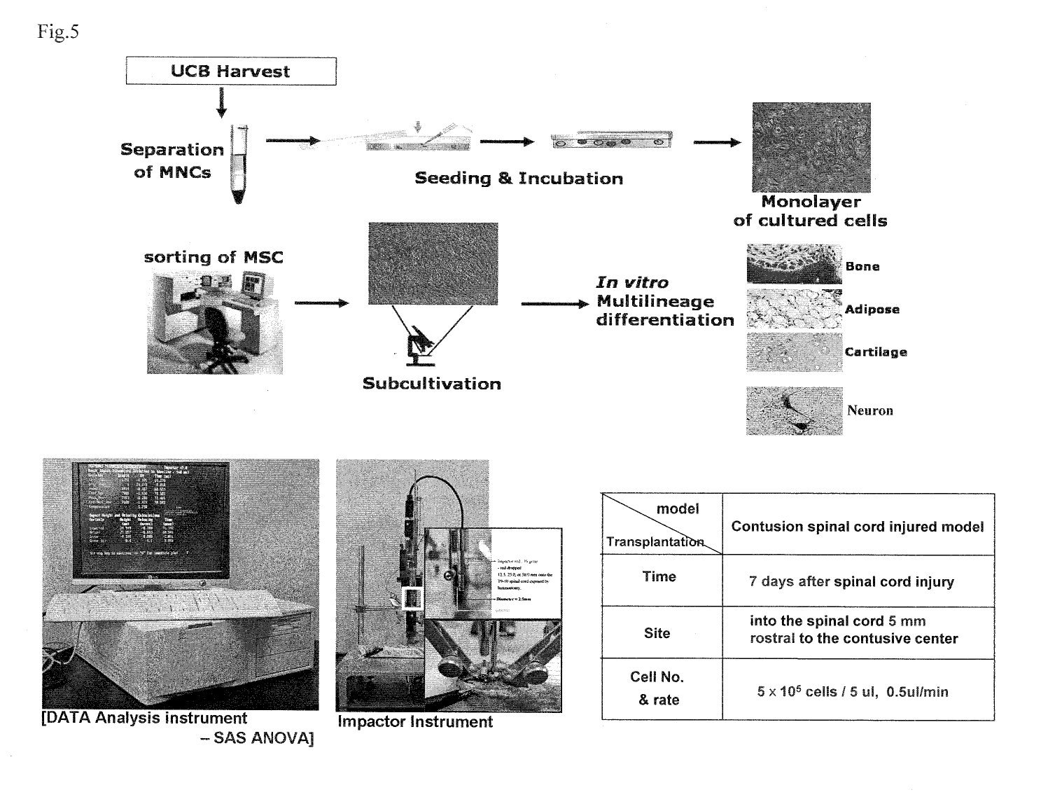

After the human umbilical cord blood-derived mesenchymal stem cell was separated in accordance with the method of FIG. 5 and transplanted, intraperitoneally BrdUrd (50 mg/kg, Sigma-Aldrich) was injected into the SD rat for labeling a newly generated cell for 14 days. Then, the checked proliferation reaction is focused on cytogenetics in which cells are generated within 14 days after transplantation of human umbilical cord blood-derived mesenchymal stem cells.

At this time, SCI was performed using a contusion model at T9 level. At the seventh day after injection, the hUCB-derived MSCs (5×10⁵ cells/5 μl) labeled with PKH26 or bisbenzimide was transplanted into a region around the injured area

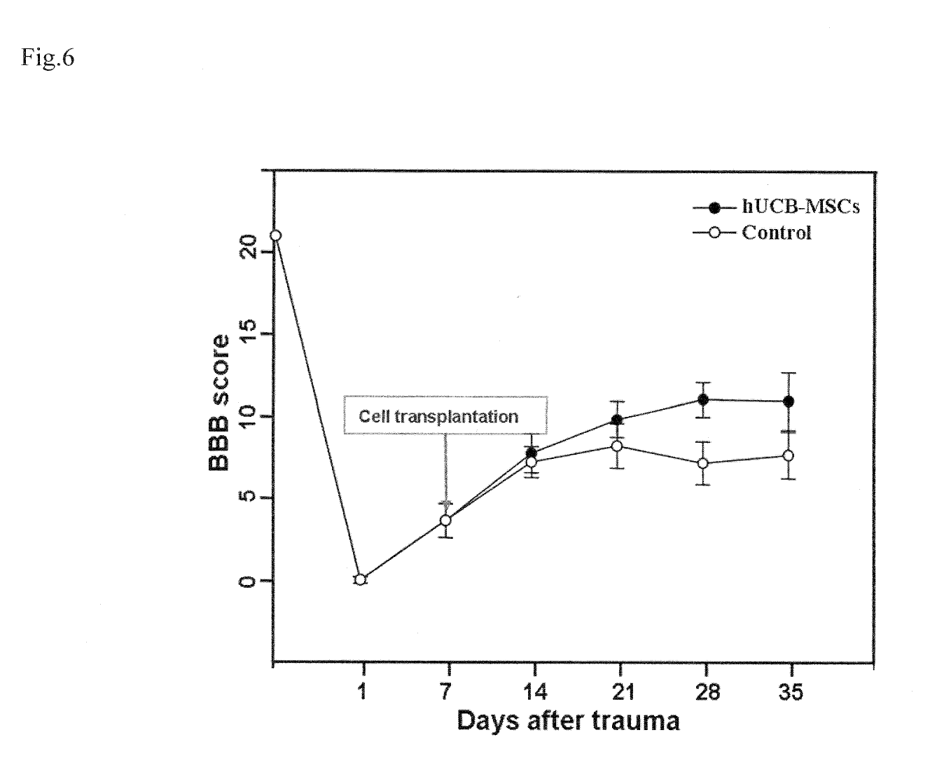

The same rotarod test as in the first embodiment was then carried out and its measured BBS grades are shown in FIG. 6.

As shown in FIG. 6, the control group treated with PBS was gradually improved so that the BBB score at the fourth week was 10.0±0.3. In contrast, a motor ability of groups transplanted with the human umbilical cord blood-derived mesenchymal stem cell significantly improved in the second week after transplantation compared to the control group treated with PBS (P<0.05). In particular, the score of the groups transplanted with the cell at the fifth week was 12.1±0.3 in the second embodiment of the present invention (score of the control group was 6.5±0.3).

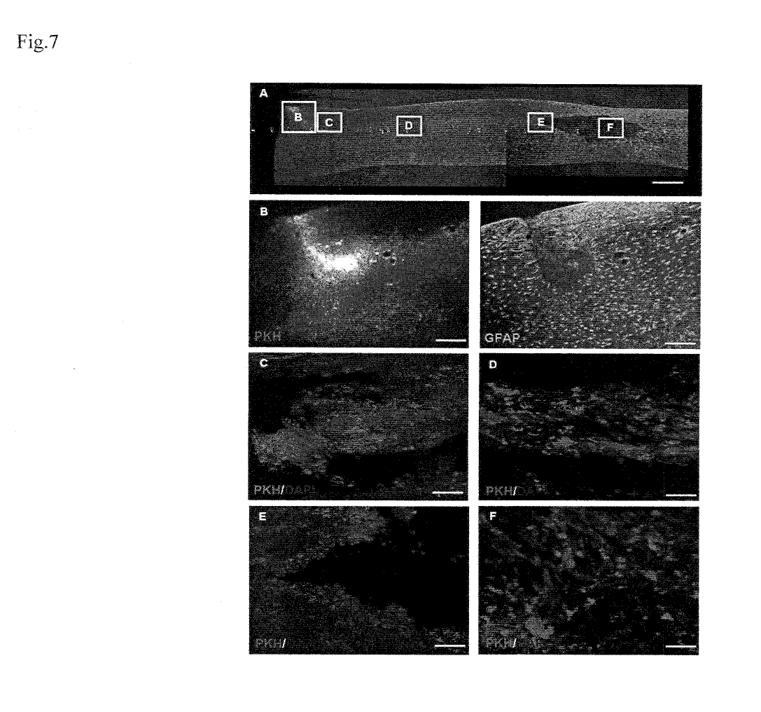

Further, immunohistofluorescence micrographs illustrating immunity stain with respect to GFAP (green fluorescence) are shown in FIG. 7, and a reference symbol A of FIG. 7 shows that the human umbilical cord blood-derived mesenchymal stem cells labeled with PKH26 at the first week after transplantation are found not only in the transplanted area but in an area around the injured area. Also, B to F of FIG. 7 show maximum magnifications of a rectangular area within area A.

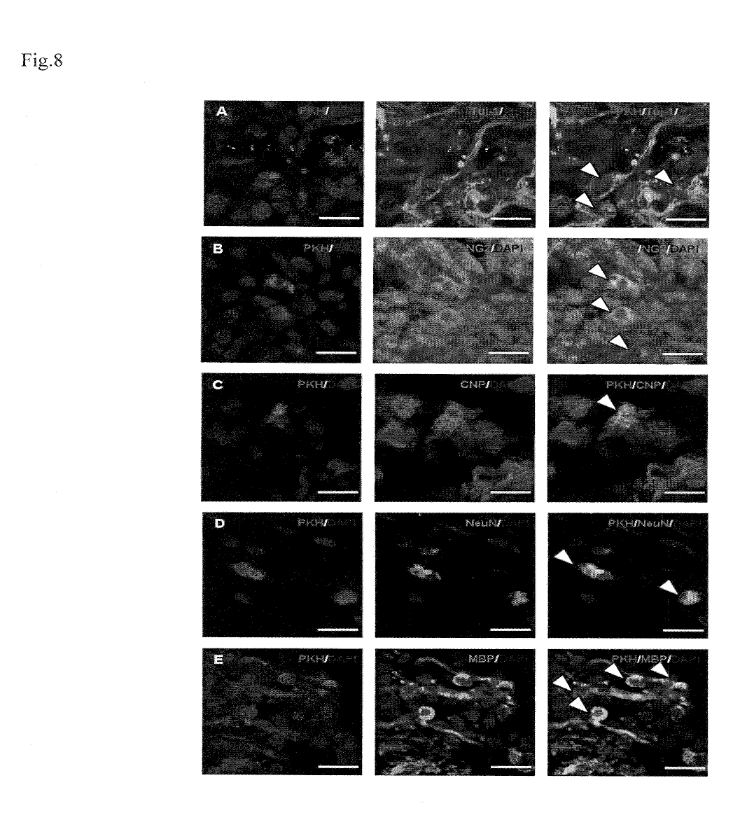

In addition, microphotographs double-labeled for a PHK26/nerve combined marker are shown in FIG. 8, and A to E of FIG. 8 show that some human umbilical cord blood-derived mesenchymal stem cells labeled with PHK26 are differentiated into nerve or oligodendrocyte.

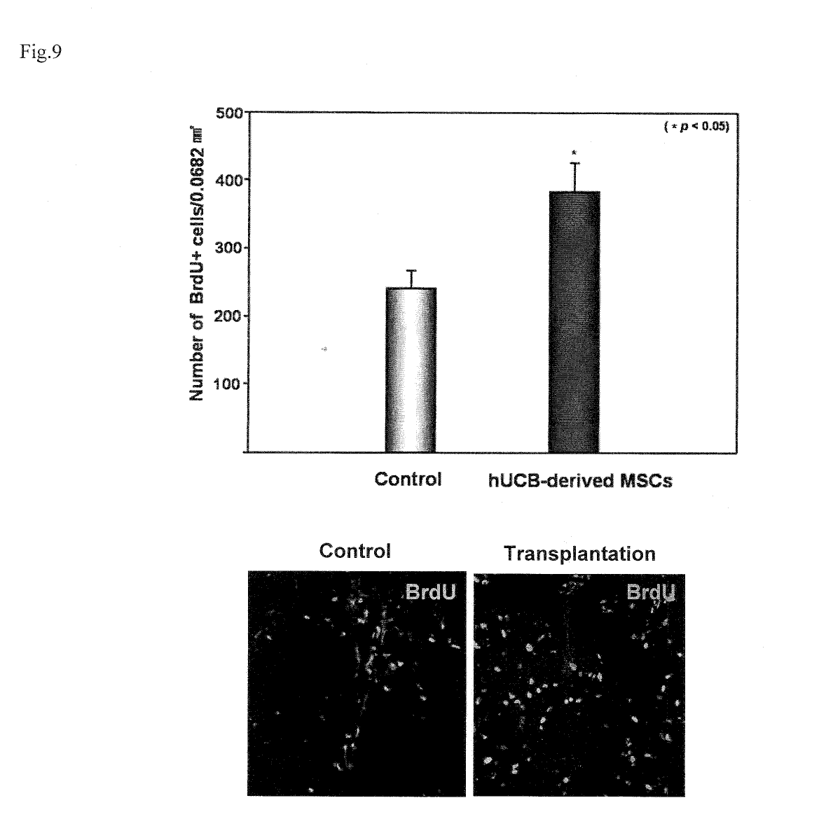

Further, quantitative analysis results of white portion cell density are shown in FIG. 9, and it can be seen that endogenous cell proliferation significantly improved within the group transplanted with a human umbilical cord blood-derived mesenchymal stem cell compared to the control group.

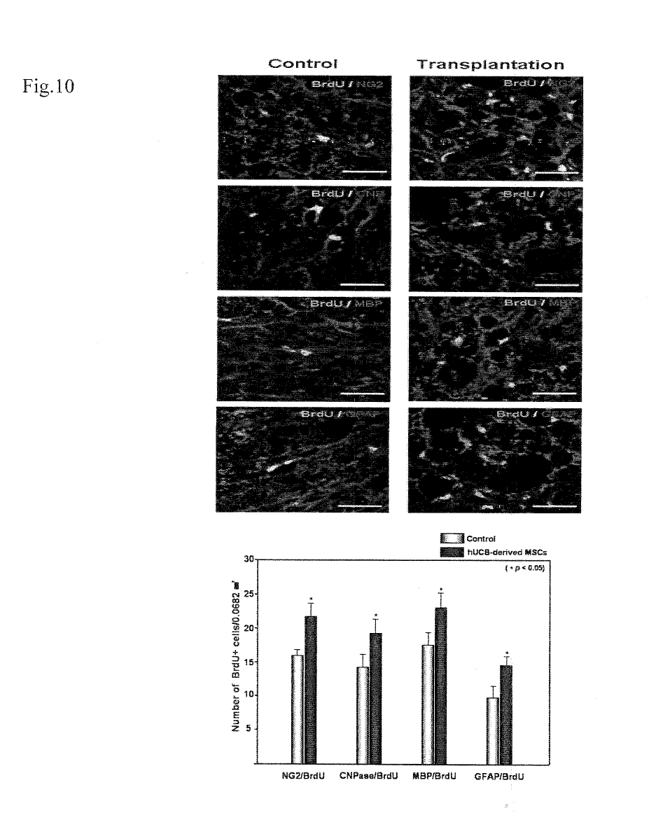

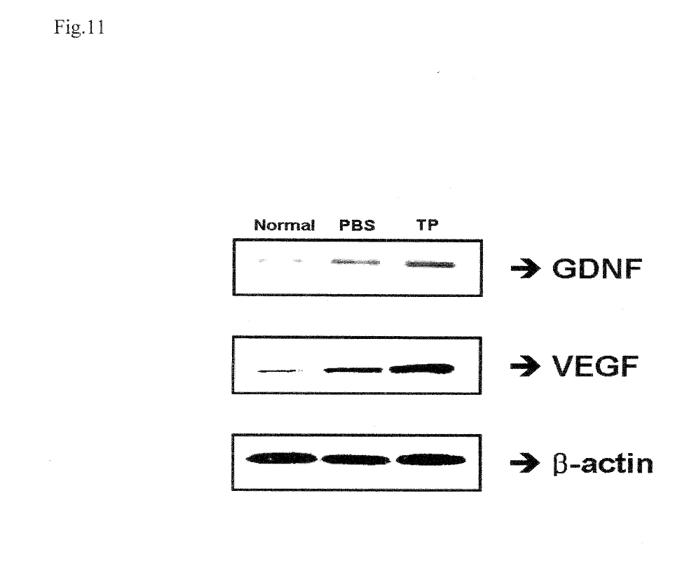

Meanwhile, distribution patterns of individual glial phenotypes within a pool of white BrdUrd at the fourteenth day after transplantation are shown in FIG. 10. And, it can be seen in FIG. 11 that transplantation of the human umbilical cord blood-derived mesenchymal stem cell increased synthesis of nerve factor expression in a traumatic spinal cord injured area.

As shown in FIG. 11, the growth factor after transplantation of human umbilical cord blood-derived mesenchymal stem cells showed stronger expression of VEGF and GDNF compared to the control group.

Results are as follows.

Firstly, compared to the control group (n=7) treated with PBS, the test group of the present invention treated with hUCB-derived MSCs (n=7) showed a significantly improved functional recovery (i.e., 6.5±0.3 vs 12.1±0.3). Also, the hUCB-derived MSCs labeled with PKH26 were usually found around the injured area, and some of them were expressed by neuronal or glial markers such as neuronal Class III β-tublin (TUJ1) neuronal nuclear antigen (NeuN) or NG2 chondroitin sulfate proteoglycan (NG2), 2,3-cyclic nucleotide 3-phosphodiesterase (CNPase) and myelin basic protein (MBP).

Secondly, labeling BrdUrd to a white rat showed that the BrdUrd-labeled endogenous nerve stem cell significantly increased marker expression to astrocytes (GFAP), NG2, CNPase and MBP within an injured area compared to the control group at the fourteenth day after transplantation with hUCB-derived MSCs. Also, the growth factor after transplantation showed strong expression of VEGF and GDNF compared to the control group.

Accordingly, intraspinal transplantation of the hUCB-derived MSCs provides differentiation of behavior recovery and neural-phenotype cells. Also, these cells stimulate proliferation and differentiation of endogenous neural stem cells and enhance expression of neurotrophic factors.

### Third Embodiment

### Effect of Human Umbilical Cord Blood-Derived Mesenchymal Stem Cell on Treatment of Stroke in a White Rat

In the present embodiment, a functional test, migration to the injured area, and differentiation into a nerve cell after UCB-MSCs transplantation will be described.

Focal cerebral ischemia was derived by intraluminal thread occlusion of middle cerebral artery (MCA). At the seventh day of transplantation, the PKH-26 labeled UCB-MSCs (3×10⁵ cells/5 μl) were transplanted to the injured cerebrum contralateral lesion of the rat.
Focal cerebral ischemia model

Sprague-Dawley male (rat weight): 230-250 g

An intraluminal filament technique (Middle cerebral artery occlusion; MCAO) was performed on transient focal cerebral ischemia.

4-0 monofilament suture; round end

120 minutes MCA occlusion; a heating pad was used to remain at 37° C.
MCAO rotarod test

Adhesive-removal test: ¢ 12, 113.1 mm² paper dot

Rotarod test; 4-40 rpm, accelerated test
preparation of brain tissue extracts

At the seventh day after MCAO, the brain of the rat was removed within 2 minutes and lyophilized in liquid nitrogen. Its tissue was homogenized in tissue 200 mg/media 1 ml (α-MEM). The homogenized solution was centrifuged (4,000 for 20 minutes). A supernatant fluid was collected, filtered (0.22 μm), and stored at −80° C.
in vitro migration assay

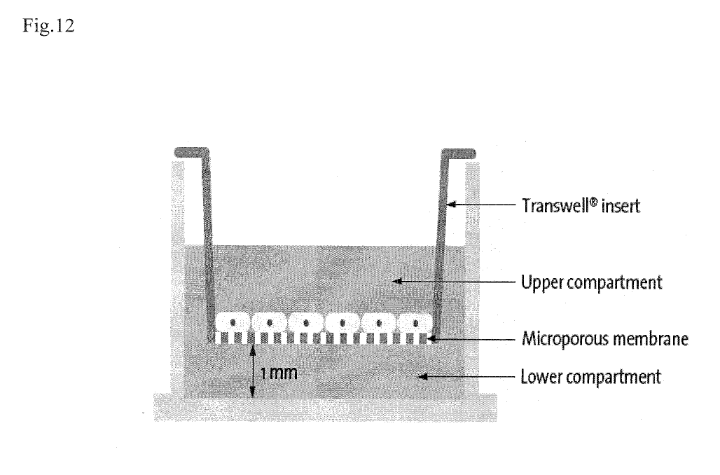

The device used is shown in FIG. 12.

transwell system (diameter of 6.5 mm, scar of 8.0 μm, corning)

upper wall: UCB-MSCs (3×10⁴ cell)

lower wall: Brain tissue extract (500 μg/ml protein)
cell transplantation

UCB-MSCs tag: PKH-26

UCB-MSCs intraverebral transplantation

period: at the seventh day after MCAO

area: epicenter (left striatum; AP+1.2/ML+2.6/DV−5.2)

Rate: 0.5 μl/min
Immunohistofluorescence

The cross-section was cut to 20 μm thickness by a cryostat. The slide was flushed with water three times by 0.01M PBS (for 5 minutes). It was blocked with a 10% normal goat serum having 0.3% triton X-100. The cross-section was incubated overnight at 4° C. using NeuN, GFAP, MBP as an antibody. The cross-section was then flushed with PBS and incubated for one hour with Alexa 488-conjugated goat anti-mouse IgG. The resulting cross-section was flushed with PBS and stained with DAPI.

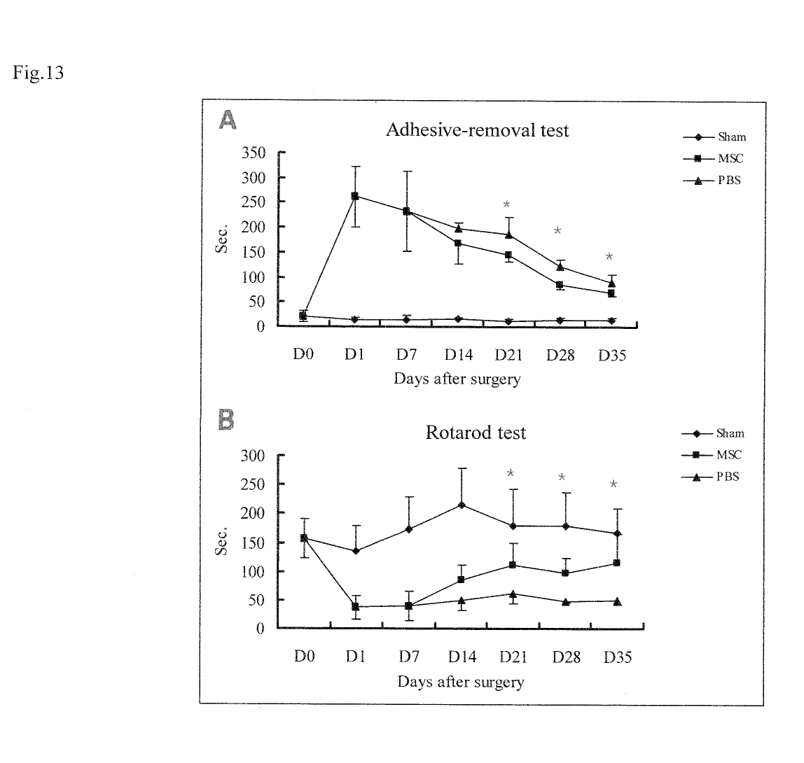

The MCAO adhesive-removal test (rotarod test) was carried out and the results are shown in FIG. 13. Referring to the same drawing, group 1 denotes Sham (n=5), group 2 denotes intracerebral transplantation of UCB-MSCs (3±0.05, n=5), and group 3 denotes intracerebral injection of PBS (n=5). The rat was killed at the 35^{th} day after MCAO. The UCB-MSCs transplanted group significantly increased over the PBS injected group in the (A) adhesive-removal test and (B) rotarod test.

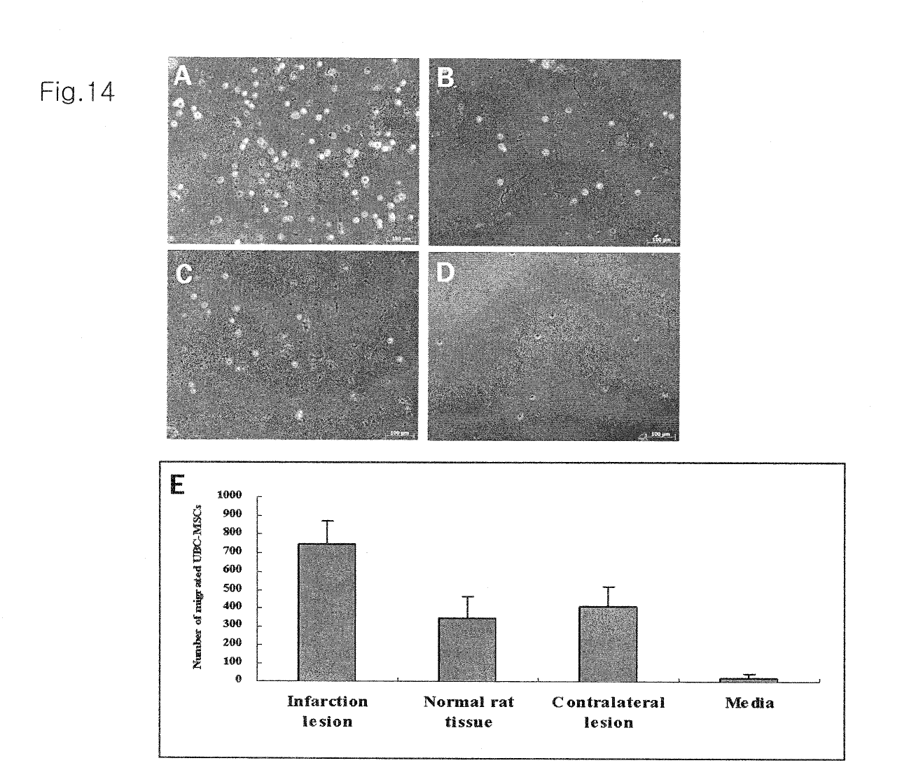

In addition, the UCB-MSCs migrated to the MCAO brain tissue extracts at the seventh day after ischemia are shown in FIG. 14: (A) Infarction lesion extracts, (B) Normal rat brain tissue extracts, (C) Contralateral lesion extracts, (D) Serum free α-MEM media, (E) The number of migrated UCB-MSCs, (A) stroke, (B,C) Non-stroke.

(E) It was confirmed that the number of UCB-MSCs migrated to the ischemic tissue extract significantly and rapidly increased over the control group.

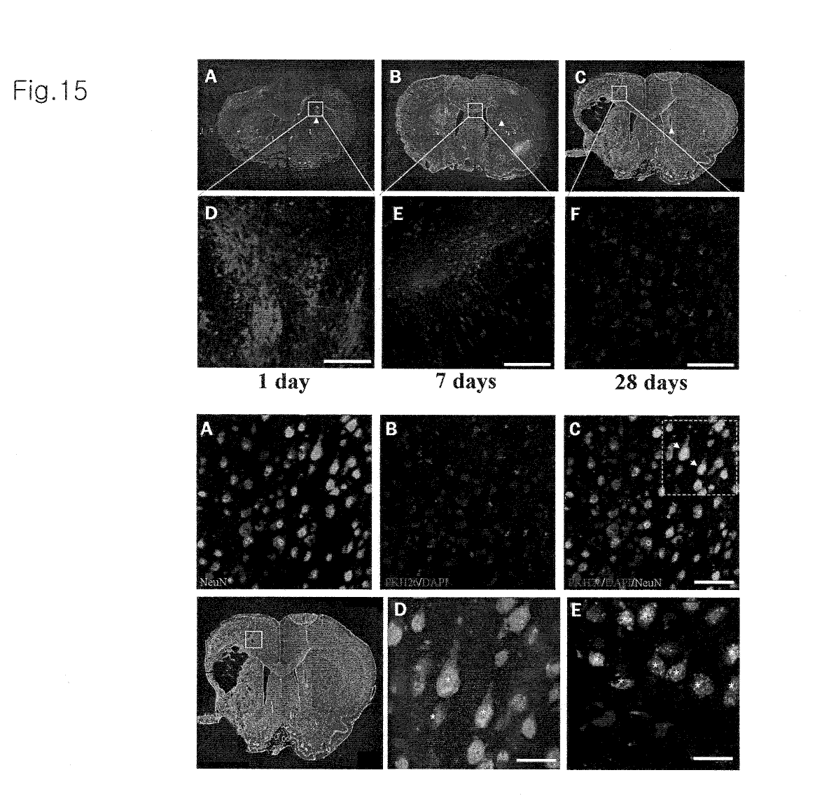

Further, it was confirmed that time serial detection UCB-MSCs migrated to an infarction lesion in FIG. 15. Referring to FIG. 15, A and D are photographs at the first day after UCB-MSCs transplantation, B and E are photographs at the seventh day after UCB-MSCs transplantation, and C and F are photographs at the 28^{th} day after UCB-MSCs transplantation.

At the first day after stem cell transplantation, the transplanted area remained. At the seventh day, the stem cell was detected at an epicenter of the corpus callosum. After 28 days, the stem cell was found at an area around the ischemia (arrow: UCB-MSCs transplanted position; red color: PKH-26; blue color: DAPI, Scale bar=50 μm).

In conclusion, (1) according to the adhesive-removal test (rotarod test), it was confirmed that the group transplanted with the MSCs showed significant improvement compared to the group injected with PBS, (2) PKH-26 labeled MSCs were detected in an ipsilateral of the injured brain of the rat at the first and fourth weeks after transplantation, and (3) a mature neuron marker NeuN was labeled on some transplanted MSCs.

According to the present invention as described above, a mesenchymal cell of the present invention isolated and incubated migrates to an injured area to be differentiated into a nerve cell or a neuroglial cell at the time of in vivo transplantation. Thus, the mesenchymal stem cell and a composition including the same can be effectively used for cell replacement therapy and gene therapy for treating diseases caused by nerve damage including stroke, Parkinson's disease, Alzheimer's disease, Pick' disease, Huntington's disease, amyotrophic lateral sclerosis, traumatic central nervous system disease and spinal cord injury.

While the invention has been particularly shown and described with reference to exemplary embodiments thereof, it will be understood by those of ordinary skill in the art that various changes in form and details may be made therein without departing from the spirit and scope of the present invention as defined by the following claims.

### CITED DOCUMENTS

1. S U Kim, Genetically modified human neural stem cells, cell therapy and regenerative medicine, Tissue Eng. Reg. Med., 1, 33 (200) 4.
2. E J Kim, J H Song, M S Kim, et al., Muscle-derived stem cells differentiate into neuronal cells for nerve regeneration, Tissue Eng. Reg. Med., 1, 41 (2004).
3. W Y Jang, S H Kim, I lee, et al., Neuvogenesis of bone marrow stromal cell using controlled release of butylated hydroxyanisole from PCLA films, Tissue Eng. Reg. Med., 2, 100 (2005).
4. K Bieback, S Kern, H Kluter, et al., Critical parameters for the isolation of mesenchmal stem cells from umbilical cord, J. Neurotra., 12, 1 (2004).
5. A Erices, P Conget, J J Minguell, et al., Mesenchymal progenitor cells in human umbilical cord blood, Br. J. Haematol., 109, 235 (2000).
6. H S Goodwin, A R Bicknese, S N Chien, et al., Mulilineage differentiation activity by cells isolated from umbilical cord blood: expression of bone, fat, and neural markers, Biol. Blood Marrow Transplant, 7, 581 (2001).
7. G Kogler, S Sensken, J A Airey, et al., A new human somatic stem cell from placental cord blood with intrinisic pluripotent differentiation potential, J. Exp. Med., 200, 123 (2004).
8. S A Kuznetsov, M H Mankani, S Gronthos, et al, Circulating skeletal stem cells, J. Cell Biol., 153, 1133 (2001).
9. P A Zuk, M Zhu, H Mizuno, et al., Multilineage cells from human adipose tissue: implication for cell-based therapies, Tissue Eng., 7, 211 (2001).
10. M Kiyama, C Radtke, J D Kocsis, et al., Remyelination of the rat spinal cord by transplantation of identified bond marrow stromal cells, J. Neurosci., 22, 6623 (2002).
11. P Ankeny, D M Mctigye, Z Gual, et al., Pegylated brain-derived neurotrophic factor shows improved distiribution into the spinal cord and stimulates locomotor activity and morphological changes after injury, Exp. Neuro, 170, 80 (2001).
12. S Garbuzova-davis, A E Willing, T Zigova, et al., Intravenous administration of human umbilical cord blood cells in a mouse model of amyotrophic lateral sclerosis: distribution, migration, and differentiation, J. Hematother. Shem Cell Res., 12 255 (2003).
13. S S Han, D Y Kang, T Mujtaba, et al., Grafted lineage-restiricted precursors differentiate exclusively into neurons in the adult spinal cord, Exp. Neuro., 177, 360 (2002).
14. S S Han, Y Liu, C Tyler-Polsz, et al., Transplantation of glial-restricted precursor cells into the adult spinal cord: survival, glial-specific differentiation and preferential migration into white matter, Glia, 45, 1 (2004).
15. M Ma, D M Basso, P Walters, et al., Behavioral and histological outcomes following graded spinal cord contusion injury in the C57B1/6 Mouse, Exp. Neuro., 169, 239 (2001).
16. M Ohta, Y Suzuki, T Noda, et al., Bone marrow stromal cells infused into the cerebrospinal fluid promote functional recovery of the injured rat spinal cord with reduced cavity formation, Exp. Neuro., 187, 266 (2004).
17. D P Ankeny, D M Mctigye, L B Kakeman, et al., Bone marrow transplantes provide tissue protection and directional guidance for axons after contusive spinal cord injury in rats, Exp. Neuro., 190, 17 (2004).
18. Q Chen, Y Long, X Yuan, et al., Protective effects of bone marrow stronmal cell transplantation in injured redent brain: Synthesis of neurotrophic factors, J. Neurosci. Res., 80, 611 (2005).
19. A Mahmood, D Lu, M Chopp, et al., Treatment f traumatic brain injury in adult rats with intravenous administration of human bone marrow stromal cells, Neurosurgery, 53, 693 (2003).
20. A Bakshi, C Hunter, S Swanger, et al., Minimally invasive delivery of stem cells for spinal cord injury: advantages of the lumbar puncture technique, J. Neurosurg. Spine, 1, 330 (2004).
21. A C Lepore, S A Swanger, A Bakshi, et al., Delivery of neural precursor cells to the injured spinal cord via intrathecal delivery at the lumbar cord, Brain Res., 1045, 206 (2005).
22. J F Ji, B P He, S T Dheen, et al., Interactions of chemokines and chemokine receptors mediate the migration of mesenchymal stem cells to the impaired site in the brain after hypoglossal nerve damage, Stem Cells, 22, 415 (2004).
23. B Ajay, L B Alissa, A S Sharon, et al., Lumbar puncture delivery of bone marrow stromal cells in spinal cord contusion: A novel method for minimally invasive cell transplantation, J. Neurotra., 23, 55 (2006).
24. M B Bunge, D D Pearse, et al., Transplantation strategies to promote repair of the unjured spinal cord, J. Rehabil. Res. Dev., 40, 55 (2003).
25. Akihiko Taguchi, Toshiniro Soma, Hidekazu Tanaka, et al. Administration of CD34+ cells after stroke enhances neurogenesis via angiogenesis in a mouse model, The Journal of Clinical Investigation, 114, 330 (2004)
26. Mary B. Newman, Alison E. Willing, et al. Stroke-induced Migration of Human Umbilical Cord Blood Cells: Time Course and Cytokines, Stem cells and Development, 14, 576 (2005)
27. S W Jeong, K Chu, K H Jung, et al. Human Neural Stem Cell Transplantation Promotes Functional Recovery in Rats With Experimental intracerebral Hemorrhage, Stroke, 34, 2258 (2003)
28. Satoshi Iihoshi, Osamu Honmou, Kiyohiro Houki, et al. A therapeutic window for intravenous administration of autologous bone marrow after cerebral ischemia in adult rats, Brain Research, 1007, 1 (2004)
29. Kazuhiko Kurozumi, Kiminori Nakamura, Tajash Tamiya, et al. Mesenchymal Stem Cells That Produce Neurotrophic Factors Reduce Ischemic Damage in the Rat Middle Cerebral Artery Occlusion Model, Molecular Therapy, 11, 96 (2005)
30. Dale Woodbury, Emily J. Schwarz, Darwin J. Prockop, et al. Adult Rat and Human Bone Marrow Stromal Cells Differentiate Into Neurons, Journal of Neuroscience Research, 61, 364 (2000)

What is claimed is:

1. A composition administered in vivo for treating nerve damage-related diseases, the composition including a mesenchymal cell as an active ingredient.

2. The composition according to claim 1, wherein the mesenchymal cell is a human umbilical cord blood-derived mesenchymal stem cell.

3. The composition according to claim 1, wherein the mesenchymal stem cell migrates to an injured area to be differentiated into a nerve cell or a neuroglial cell.

4. The composition according to claim 1, wherein the nerve damage-related disease is one selected from the group consisting of a stroke, Parkinson's disease, Alzheimer's disease, Pick's disease, Huntington's disease, amyotrophic lateral sclerosis, traumatic central nervous system disease and a spinal cord injury.

5. The composition according to claim 1, wherein the nerve damage-related disease is a stroke or a spinal cord injury.

6. A composition administered in vivo having a nerve damage protection effect and comprising a mesenchymal cell as an active ingredient.

7. A composition administered in vivo having a nerve damage regeneration effect and comprising a mesenchymal cell as an active ingredient.

8. The composition according to claim 1, wherein the in vivo administration is rostral administration in injured areas.

9. A method of treating nerve damage-related diseases by administering a therapeutically effective amount of a composition according to claim 1.

10. The method according to claim 9, wherein a mesenchymal cell is a human umbilical cord blood-derived mesenchymal stem cell.

11. The method according to claim 9, wherein the mesenchymal stem cell migrates to an injured area to be differentiated into a nerve cell or a neuroglial cell.

12. The method according to claim 9, wherein the nerve damage-related disease is one selected from the group consisting of a stroke, Parkinson's disease, Alzheimer's disease, Pick's disease, Huntington's disease, amyotrophic lateral sclerosis, traumatic central nervous system disease and a spinal cord injury.

13. The method according to claim 9, wherein the nerve damage-related disease is a stroke or a spinal cord injury.

14. The method according to claim 1, wherein the in vivo administration is rostral administration in injured areas.

15. The method according to claim 9, wherein the composition is for treating mammals.
